(19) United States
(12) Patent Application Publication
(10) Pub. No.: US 2007/0264352 A1
(43) Pub. Date: Nov. 15, 2007

(54) Biostimulate and tonic solution for massage (solution "Biplatvirs")

(76) Inventor: Yakov Brilman, Voorhees, NJ (US)

Correspondence Address: FLASTER/GREENBERG P.C.;8 PENN CENTER, 1628 JOHN F. KENNEDY BLVD., 15TH FLOOR, PHILADELPHIA, PA, 19103, US

(21) Appl. No.: 11/432,024

(22) Filed: May 10, 2006

(51) Int. Cl. A61K 35/413; A61K 35/50

(52) U.S. Cl. 424/528; 424/725

(57) ABSTRACT

The composition “BIPLATVIRS” is a dark brown liquid, intended for external use by sanative massage. The composition possesses tonic and biostimulant characteristics. The composition can be a solution which consists of a mixture in which bile from young, sound animals is combined with extract of the placenta, extract from the thymus gland, extract of rhodiola fluid, and tincture of sterculia, along with a complex of vitamins.

### BACKGROUND OF THE INVENTION

1. Field of the Invention

The invention pertains to the area of medicine and is designed for external use by sanative massage.

2. Description of Related Art

This invention was developed during the course of my medical work. I used “Bile Medical” for treatment of patients with systemic musculoskeletal diseases by massage of the back and spine. Patients, after such massage, noted a flow of vigor. Its toning effect improved the patient's general condition. This was a result, not only of massage, but also the combination of it with bile, which contains physiological active substances—hormones, vitamins, ferments, and others.

It is known that bile lowers the surface pull of cellules, increases permeability of their membranes, and is a biological stimulant.

Herewith also consider that the bile contains concentrations of the ferment tansaminase (in amounts 100 times greater than in blood serum). It is know that transaminase possesses catalysis characteristics, increasing its activity in blood serum by pathological conditions, accompanied by a destruction process (such as a myocardial heart attack, viral hepatitis and others).

The inunction of the bile on the skin of the organism creates an intradermal deposit that contains the bite that transaminases and enters into the blood with hormones, vitamins, ferments, etc. that are included in the bile.

### BRIEF SUMMARY OF THE INVENTION

The proposed solution “BIPLATVIRS” is a biostimulant and tonic preparation. It is founded on using bile from young, sound animals (such as calves and piglets). This bile, aside from having biostimulant and tonic characteristics, also enlarges permeability of the cells of the organism, so that the proposed solution may be used for introduction to an organism of additional material. Bile is offered in combination with a solution with some preparations (extract of the placenta, extract of the thymus gland, vitamins, extract of rhodiola fluid and tincture sterculia), which also possess biostimulant and tonic characteristics for the reason of reinforcement of the effect of the bile.

The contents from the bile also promote creation of a slippery slithering movement of the hands on body surfaces where the skin is being massaged.

The invention discloses a mixture of medicinal material based on the use of bile as a biostimulant and tonic preparation in combination with other medicinal material which also possess like characteristics that reinforce this effect.

The invention discloses a method for making such a composition using bile from calves and piglets in equal parts (1:1 ratio); and alternately by including in the composition a blend of 5% extract of the placenta, 5% extract from the thymus gland, 3% extract of rhodiola fluid, 3% tincture of sterculia; and alternately by using a preservative such as tricresol.

The invention discloses a method for using rhodiola extract to hide the specific scent of bile in this composition.

The invention discloses a method for applying the composition by way of massage as, for example, a liniment on the skin.

### DETAILED DESCRIPTION OF THE INVENTION

The invention is a stimulant and tonic preparation based on bile from young, healthy animals such as calves and piglets.

A solution including bile with additional material also possessing tonic characteristics added for reinforcement is included herein. The reason for using bile, which is an efficient biostimulant in massage, in treatment of weakened, sick, or gravely suffering patients is due to the process of aging of the organism. In connection with this, I am offering a new combination of medicinal material to be taken externally, used (in massage) as a facility tonic and to stimulate improvements in the state of the health of the sick person.

In order to reinforce the tonic and biostimulant influencing bile, and providing for the possibility of its use, and its characteristic of increasing the permeability of material through the skin, I am offering a combination created by its effect, with some other means, which also provide biostimulant and tonic characteristics: including extract of the placenta (water extract of human placenta preserved through cold which is used for injections as a biological stimulant for some diseases); water extract from the thymus gland (filtrated from the suspension of a thymus gland of a young animal (such as a calf) in isotonic solution of sodium chloride preserved through cold (this extract is also entered into the proposed solution as a biostimulator), with consideration that hormones from this gland regulate the formation and functioning mechanism's cellular immunity; and, for the same purpose, a complex of vitamins (A; B₁; B₂; B₆; C; P) is also added to bile.

To the tonic material, add tincture of sterculia and liquid extract of rhodiola fluid (which in addition to any material added for smell, give an aromatic scent to the solution).

In one preferred embodiment, the composition, which is identified herein as “BIPLATVIRS” mixture and which is offered for use as a biostimulant and tonic solution for massage, contains the following:
1) a mixture of bile of calves and piglets (1:1)—82.0%
2) aqueous (water) extract of the placenta—5%
3) aqueous (water) extract from the thymus gland—5%
4) Vitamins:
   a) Retinol acetate or Retinol palmitate—0.02%
   b) Thiamine chloride or Thiamine bromide 0.02%
   c) Riboflavin 0.02%
   d) Pyridoxine hydrochloride 0.02%
   e) Ascorbic Acid 0.9%
   f) Rutin 0.22%
5) extract of Rhodiola fluid—3% (it is also a smelling/scent material)
6) tincture of Sterculia—3%
7) natrium chloride solution—0.5%
8) tricresol—0.3% or another preserving material.

Note that it is necessary to take or use the bile in a sterile condition for prevention of bacterial contamination.

This above solution is used for inunction in the skin of the back and spine for about 10-15 minutes for performing sanative massage. Preferably, the sanative massage should be performed once daily, during a determined time, individually for each patient at the discretion of the doctor in charge of the patient's case.

Contents of bile also promote slippery, slithering movements of the hands on surfaces of the skin in the massaged areas of the body.

The solution is quickly absorbed by the skin of the patient and within a few minutes becomes dry. For 12 hours after such a massage, the solution should not be washed off the skin.

The biostimulate and tonic solution should be applied externally. The base of the preparation uses the bile from a young animal or animals to increase intradermal permeability of the contents of the solution, and has a biostimulant and tonic influence or effect on the organism. For reinforcement of the biostimulant and tonic effects of the bile, the solution includes material possessing like characteristics.

The proposed solution is capable of finding broad uses for recovery and rejuvenation of an organism through its use in sanative massage alone or with other methods. It can also be used by weakened, sick people after diseases, operations, or conditions associated with the process of aging of the organism.

The inventive solution can also be used in cosmetics as a biostimulant and tonic preparation for the skin (removing wrinkles, etc.)

Contraindications for the solution include hypertension, severe heart and/or vascular disease, skin disease, individual intolerance, malignant new formations.

1-7. (canceled)

8. A therapeutic composition for external massage of an animal comprising:
a solution of animal bile;
a solution of human placenta extract;
a solution of thymus gland extract; and
a composition of vitamins,
wherein the animal bile solution is obtained from young animals.

9. The composition according to claim 8, wherein the animal bile is a combination of calf bile and piglet bile in a 1:1 ratio by weight.

10. The composition according to claim 9, wherein the composition further includes additives for an aromatic scent.

11. The composition according to claim 10, wherein the composition further includes a preservative solution.

12. The composition according to claim 11, wherein the animal bile solution is 82% by weight of the composition, the human placenta solution is 5% by weight of the composition, the thymus gland solution is 5% by weight of the composition, the vitamin composition is 1.2% by weight of the composition, the scent additive is 6% by weight of the composition and the preservative solution is 0.8% by weight of the composition.

13. The therapeutic composition according to claim 10, wherein the scent additives are extract of Rhodiola fluid and tincture of Sterculia.

14. The therapeutic composition according to claim 11, wherein the preservatives are natrium chloride solution and tricresol.

15. The therapeutic composition according to claim 12, wherein the vitamin composition comprises vitamin A, B₁, B₂, B₆, C and P.

16. A method for external massage of an animal by use of a therapeutic composition comprising the steps of:
directing the therapeutic composition to the external skin of an animal, wherein an animal bile solution comprises 82% by weight of the composition, a human placenta solution is 5% by weight of the composition, a thymus gland solution is 5% by weight of the composition, a vitamin composition is 1.2% by weight of the composition, a scent additives is 6% by weight of the composition and a preservative solution is 0.8% by weight of the composition; and
messaging the therapeutic composition into the external skin of an animal for about 10 to 15 minutes once daily until the pain is eliminated.
